(19) United States
(12) Patent Application Publication
(10) Pub. No.: US 2013/0302885 A1
(43) Pub. Date: Nov. 14, 2013

(54) Method of Forming Cell Spheroids Cultured in Serum-Free Manner on Nanoscale Coatings of Hyaluronic Acid with High Molecular Weight

(71) Applicants: Jui-Yang Lai, Guishan Township (TW); I-Hao Tu, Tainan City (TW); Ting-Chun Yu, Guishan Township (TW)

(72) Inventors: Jui-Yang Lai, Guishan Township (TW); I-Hao Tu, Tainan City (TW); Ting-Chun Yu, Guishan Township (TW)

(21) Appl. No.: 13/470,429

(22) Filed: May 14, 2012

(51) Int. Cl. C12N 5/071

(52) U.S. Cl. 435/325

(57) ABSTRACT

A method of forming cell spheroids cultured in a serum-free manner on nanoscale coatings of hyaluronic acid with high molecular weight is disclosed. Corneal stromal cells are cultured on the hyaluronic acid coatings with nanotopography in the serum-free manner. Experimental results show that the cells cultured on the hyaluronic acid coatings (1.1-1.7 nm) increase cell-cell interaction, and when the cells form three-dimensional spheroids, they have higher biological synthetic capabilities and can secret more extracellular matrix, which has potential applications in corneal stromal tissue reconstruction.

### FIELD OF THE INVENTION

The present invention relates to a field of cell culture, and more particularly to a method of forming cell spheroids cultured in a serum-free manner on nanoscale coatings of hyaluronic acid with high molecular weight. Clinically, it can effectively reconstruct damaged corneal stroma and overcome the drawback associated with the shortage of donor corneas.

### BACKGROUND OF THE INVENTION

Currently, full-thickness penetrating keratoplasty (PK) is the most commonly used method to treat corneal disease. The method basically removes the damaged corneal tissue and substitutes with a normal cornea from a donor to improve the vision. However, the full-thickness corneal transplant surgery often causes complications, such as poor wound closure, immune rejection, postoperative infection, neovascular, glaucoma and cataracts. Also, postoperative recovery is usually slow, and the patient may have irregular astigmatism to affect the corrected vision. Therefore, researchers started to study tissue engineering of corneal stroma, which is expected to replace cornea transplant.

Recently, researchers tried to use materials that can directly induce cells to form spheroids. In 2006, Lin et al. induced cells to form spheroids to treat vitiligo using melanocyte cultured on chitosan coatings. In 2007, Lin et al. prepared the chitosan/nylon blends in different proportions to explore cell attachment and formation. The results showed that with the increase of nylon in the mixture, the cells are more likely to attach to the surface and reduce cellular aggregation [Sung-Jan Lin, Shiou-Hwa Jee, Wen-Chu Hsiao, Hsin-Su Yu, Tsen-Fang Tsai, Jau-Shiuh Chen, Chih-Jung Hsu, Tai-Horng Young. Enhanced cell survival of melanocyte spheroids in serum starvation condition. Biomaterials, 2006, 27, 1462-1469] [Sung-Jan Lin, Wen-Chu Hsiao, Shiou-Hwa Jee, Hsin-Su Yu, Tsen-Fang Tsai, Juin-Yih Lai, Tai-Horng Young. Study on the effects of nylon-chitosan-blended membranes on the spheroid-forming activity of human melanocytes. Biomaterials, 2006, 27, 5079-5088]. In 2009, Lee et al. cultured mesenchymal stem cells (MSCs) on thermo-responsive hydrogel to induce the cell to form spheroids [Wen-Yu Lee, Yu-Hsiang Chang, Yi-Chun Yeh, Chun-Hung Chen, Kurt M. Lin, Chieh-Cheng Huang, Yen Chang, Hsing-Wen Sung. The use of injectable spherically symmetric cell aggregates self-assembled in a thermo-responsive hydrogel for enhanced cell transplantation. Biomaterials 2009, 30, 5505-5513]. In the same year, Chen et al. cultured corneal stromal cells on the chitosan coatings, and the results showed that the formed cell spheroids can maintain their phenotype [Yi-Hsin Chen, I-Jong Wang, Tai-Horng Young. Formation of keratocyte spheroids on chitosan-coated surface can maintain keratocyte phenotypes. Tissue Engineering—Part A, 2009, 15, 2001-2013]

The prior arts suggest that material property is an important factor to induce cells to aggregate. Also, when different cells form spheroids, the cell growth and differentiation may be further affected. For example, liver spheroids secrete albumin; PC12 cell spheroids can regulate cells and secrete substantial amount of dopamine; neural stem cell spheroids can maintain stem cell characteristics. Thus, cell spheroids can bring new opportunities in the medical fields of reconstruction.

### SUMMARY OF THE INVENTION

The present invention provides a method of forming cell spheroids cultured in a serum-free manner on nanoscale coatings of hyaluronic acid with high molecular weight. High molecular weight hyaluronic acid has more negative charges, and possesses higher surface roughness and hydrophilicity when being processed to form nanoscale coatings. These properties lower cell attachment and cell-matrix interaction. Cell behavior is determined by the environment initially interacts with the cells. Experimental results show that cells cultured on the hyaluronic acid coatings (1.1-1.7 nm) are beneficial for cell-cell interaction, so the cells are easy to aggregate to form cell spheroids and maintain mitotically quiescent state. After the cells form three-dimensional cell spheroids, their biosynthetic capability becomes higher to secrete more extracellular matrix. When corneal stromal cells are cultured on TCPS and HA35, the cells are single-layered, while the cells cultured on HA360 and HA1500 form multicellular spheroids. These cell spheroids have higher keratocan and lumican and lower biglycan gene expression level comparing with monolayered cells. In addition, larger cell spheroids have higher gene expressions of ALDH and Nestin, which shows that the cells can maintain high transparency and better self-renewal capability.

Effect: The present invention provides a method of forming cell spheroids cultured in a serum-free manner on nanoscale coatings of hyaluronic acid with high molecular weight. The cell spheroids cultured on the hyaluronic acid coatings with high molecular weight have following characteristics: (1) good viability; (2) mitotically quiescent state; (3) proper phenotype; and (4) good biosynthetic capability. When applying in repairing corneal stroma, only cell spheroids are necessary to be transplanted, which can solve the problem of immune rejection and biocompatibility for other biomaterials. Also, comparing with cell suspensions, the cell spheroids can significantly increase the therapeutic efficiency in the tissue reconstruction.

### BRIEF DESCRIPTION OF THE DRAWINGS

FIG. 1 illustrates a process flow of one embodiment of the present invention.

FIG. 2 illustrates a surface charge diagram of hyaluronic acid with different molecular weights.

FIG. 3 illustrates a surface topographic analysis diagram of hyaluronic acid coatings with different molecular weights.

FIG. 4 illustrates a microscopic observation diagram of surface hydrophilic/hydrophobic characteristics of (A) TCPS, (B) HA35, (C) HA360 and (D) HA1500.

FIG. 5 illustrates a microscopic observation diagram of the corneal stromal cells attached to the hyaluronic acid coatings of different molecular weights: (A) TCPS, (B) HA35, (C) HA360, (D) HA1500, wherein LM-h1 means at the time period of one hour under optical microscope, the LM-h8 means at the time period of eight hours under optical microscope, and SEM-h8 means at the time period of eight hours under scanning electron microscope.

FIG. 6 illustrates cell adhesion ratio of the corneal stromal cells on hyaluronic acid coatings of different molecular weights.

FIG. 7 illustrates cell number of the corneal stromal cells grown on hyaluronic acid coatings of different molecular weights.

FIG. 8 illustrates sizes of corneal stromal cell spheroids on the hyaluronic acid coatings of different molecular weights.

FIG. 9 illustrates collagen analysis of corneal stromal cell spheroids on the hyaluronic acid coatings of different molecular weights.

FIG. 10 illustrates GAG analysis of corneal stromal cell spheroids on the hyaluronic acid coatings of different molecular weights.

FIGS. 11A and 11B illustrate gene expression levels of corneal stromal cell spheroids on the hyaluronic acid coatings of different molecular weights.

FIG. 12 illustrates a time-course slit-lamp observation.

FIG. 13A illustrates an observation of corneal thickness versus time.

FIG. 13B illustrates an observation of intraocular pressure versus time.

FIG. 14A illustrates a six-group model of corneal analysis.

FIG. 14B illustrates a six-group model of corneal optical transmittance.

FIG. 15 illustrates a fluorescent microscopic observation diagram of rabbit corneal stroma received PKH26-labeled corneal stromal cell suspensions and spheroids.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

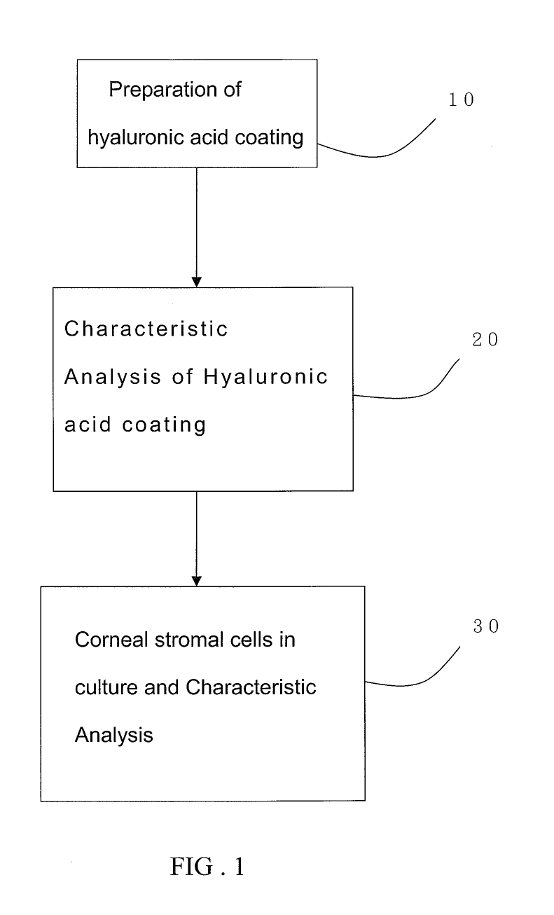

Referring to FIGS. 1 to 15, the present invention provides a method of forming cell spheroids cultured in serum-free manner on nanoscale coatings of hyaluronic acid with high molecular weight. Furthermore, the present invention includes culturing keratocytes on nanoscale coatings of hyaluronic acid with different molecular weights and investigating the behavior. As shown in FIG. 1, the steps include:

Step (10): Preparing coatings of hyaluronic acid with different molecular weights;

Step (20): Analyzing each hyaluronic acid coatings regarding surface roughness, surface potential and water contact angle; and

Step (30): Culturing corneal stromal cells on the nanoscale hyaluronic acid coatings, conducting cell adhesion analysis, cell proliferation analysis, quantitative spheroid size and number analysis, cell extracellular matrix analysis and gene expression analysis; and maintaining the growth of the cell to form cell spheroids.

To prepare the hyaluronic acid coatings, the hyaluronic acid with three different molecular weights (35,000; 360,000; 1,500,000 Dalton) was dissolved in deionized water to make an aqueous solution of 1 mg/mL concentration. Subsequently, 1 mL solution was coated onto 24-well non-tissue culture plates (Falcon 351147), and the sample was placed into a 25° C. oven with constant temperature for three days until the surface is completely dry.

For the following embodiments, the experimental data are obtained according to “mean±standard error of the mean (SEM).” All data are analyzed by one-way ANOVA to access the differences between the groups. If the statistical analysis is p<0.05, there is statistical significance.

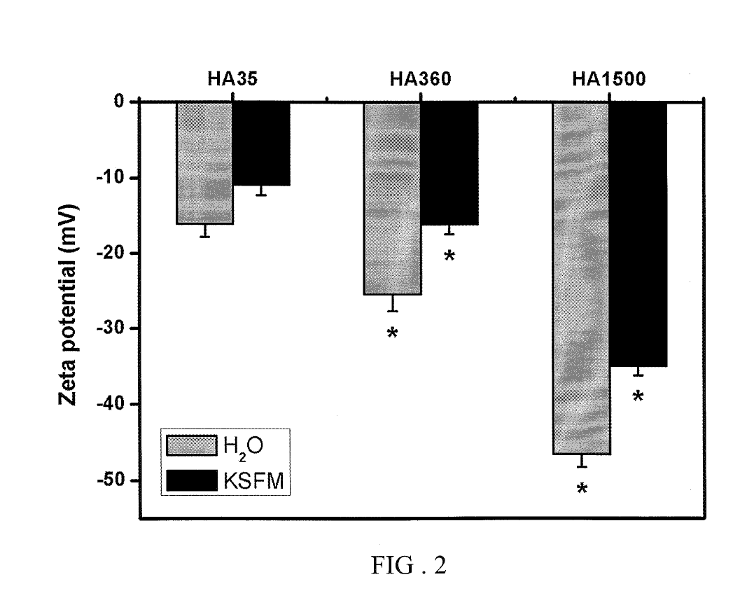

Surface potential analysis: the hyaluronic acid is dissolved in deionized water of pH 7.0, or the culture medium of pH 7.4 to prepare the solution of 1 mg/mL concentration. Take 1.0 mL of hyaluronic acid solution to conduct Doppler microelectrophoresis analysis of surface charge of different molecular weights. FIG. 2 (*P<0.05; n=4) shows surface charge of hyaluronic acid of different molecular weights (35,000; 360,000; 1,500,000 Dalton) that dissolved in deionized water and KSFM culture medium. Since the hyaluronic acid has a carboxylic functional group, the surface charge of all test samples is negative. The surface charges in deionized water are HA35 (−16.0±1.8 mV), HA360 (−25.4±2.3 mV), and HA1500 (−46.5±1.7 mV), respectively. The surface charges in KSFM culture medium are HA35 (−10.9±1.4 mV), HA360 (−16.1±1.3 mV), and HA1500 (−34.9±1.3 mV), respectively. In addition, increasing the molecular weight of hyaluronic acid can increase the negative charge on the surface because the hyaluronic acid with higher molecular weight has more carboxylic acid functional groups (—COOH). It is noted that if the solvent is changed to the culture medium, the negative surface charge is far lower than that in the deionized water.

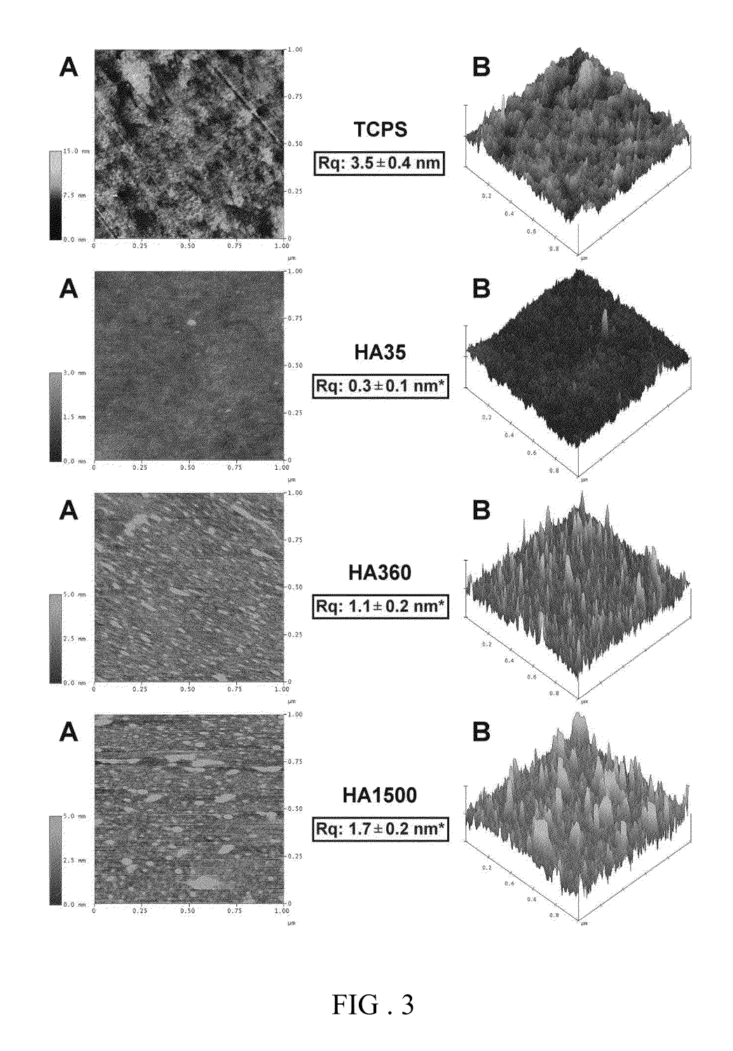

Surface roughness analysis: assess the surface roughness of hyaluronic acid with different molecular weights. A probe is clipped at a tip holder and the sample is fixed on an atomic force microscope (AFM). After the focus is adjusted to clearly see the film, the probe is slowly lowered to the surface of the coatings, and a laser spot is adjusted to a probe cantilever so that the light source can totally reflect to a photo detector. The surface can be observed with a surface tapping mode, and the scanning speed is 0.4 Hz. AFM images are recorded with the scan size of 1 μm×1 μm, and the root-mean-square roughness (Rq) can be calculated. FIG. 3 (*P<0.05; n=3) shows the surface morphology of the hyaluronic acid with different molecular weights, and the results show that the hyaluronic acid with higher molecular weight has higher surface roughness (the surface roughness of HA35, HA360, HA1500 is 0.3±0.1 nm; 1.1±0.2 nm; 1.7±0.2 nm, respectively). In other words, the surface roughness of the nanoscale hyaluronic acid coatings increases with the molecular weight of the hyaluronic acid, and this surface phenomenon is closely related to the chain length of the hyaluronic acid. For a substrate, when a highly hydrophilic acid is grafted onto another interface of a material, there are more interfacial interactions between the branches of the hyaluronic acid and the interface, and the surface roughness increases accordingly. Also, when the surface roughness increases, the hydrophilicity increases as well, which makes the cells unlikely to attach to the surface, and the cells are difficult to differentiate.

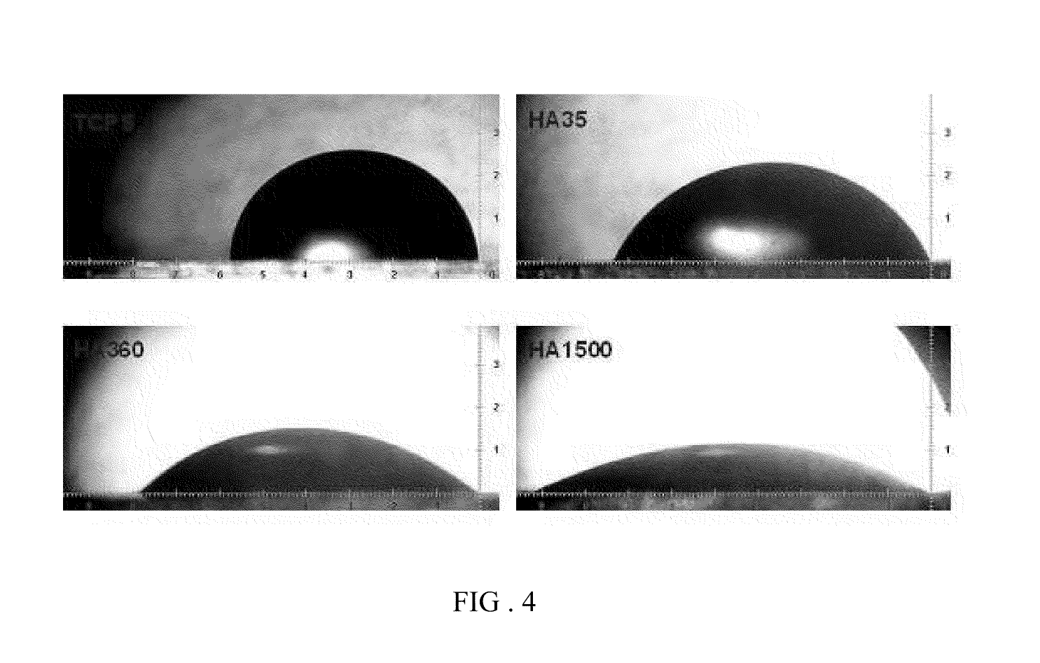

Surface contact angle analysis: the contact angle of hyaluronic acid is measured by a sessile drop method to assess surface wettability. The hyaluronic acid is coated on a contact angle goniometer, and deionized water of 2 μL is dripped onto the hyaluronic acid surface for one minute at 25° C. Subsequently, measure an angle between the baseline of the droplet and the liquid/solid/gas (three phases) contact point, repeat the measurement for six times at different locations and calculate the average value. FIG. 4 shows hydrophilic/hydrophobic analysis of the hyaluronic acid with different molecular weights, and the results show that when the hyaluronic acid is coated on the culture dish, the water absorption interface increases with the increase of the molecular weight, meaning that the higher the molecular weight, the higher the hydrophilicity. In addition, the contact angles of HA35, HA360, HA1500 are 58.33±0.73°, 48.85±0.75° and 34.06±1.24°, respectively.

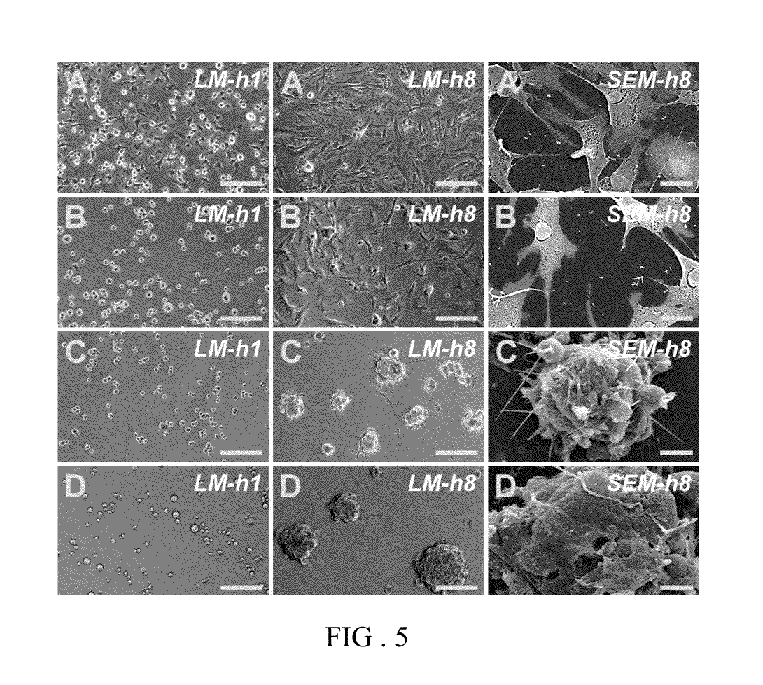

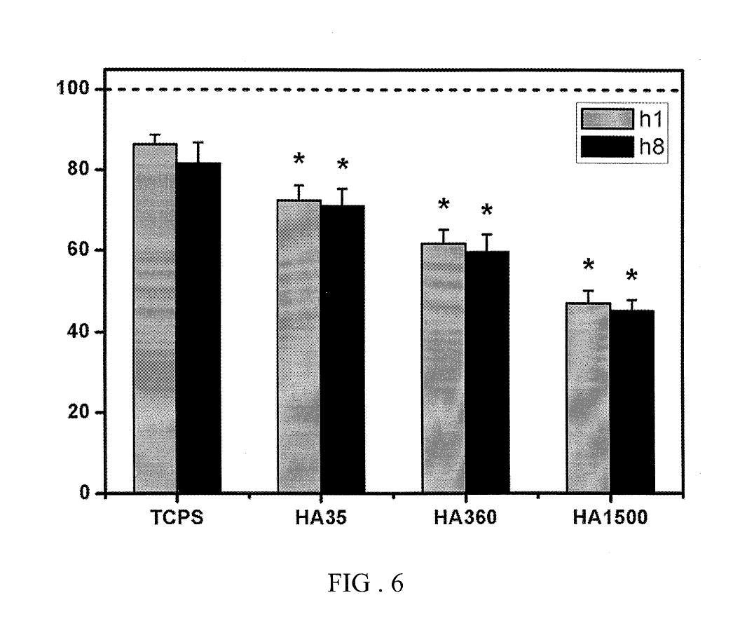

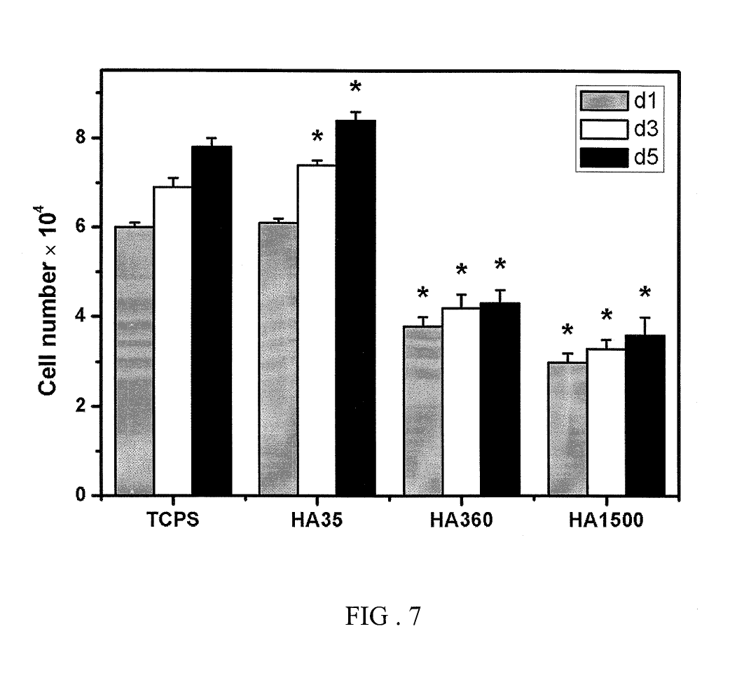

Cell attachment and proliferation: before cell culture, the hyaluronic acid coatings with different molecular weights (35,000; 360,000; 1,500,000 Dalton) are placed under UV sterilizer for two hours. Subsequently, add 1 mL corneal stromal cells (5×10⁴ cell/mL) and incubate at 37° C. Observe cell attachment under a microscope at the first and eighth hour, and analyze the quantity of cell attachment. For the experiment of cell proliferation, cells are cultured on the hyaluronic acid coatings for 1, 3, and 5 days, and observe and analyze cell proliferation and cell growth. The results show that the absorption value is proportional to the cell activity. FIG. 5 shows cell attaching to the hyaluronic acid coatings with different molecular weights. When the corneal stromal cells are attached for one hour, the result on TCPS shows good adhesion characteristics, and under an optical microscope, it is observed that many cells have begun to attach to the interface comparing with the interface with different molecular weights. The results for eight-hour attachment show that a two-dimensional culture status has formed at the interface of TCPS and HA35. A three-dimensional cell spheroid is formed at the interface of HA360 and HA1500, respectively. Comparing the cell spheroid of HA1500 with HA360, since HA1500 has higher surface negative charge and surface roughness, the effect of cell-extracellular matrix is weakened, while the effect of cell-cell interaction is strengthened to increase cell aggregation. Also, the corneal stromal cells attached to TCPS have good adhesion effect as HA35 and the cells are dendritic, observed under a scanning electron microscope. When cells are cultured on HA360 and HA1500 hyaluronic acid coatings, the cells started to aggregate to form spheroids and the aggregation effect increases when the molecular weight increases. Therefore, culturing the corneal stromal cells on HA360 and HA1500 hyaluronic acid coatings helps the cells form a three-dimensional structure that is similar to a biological environment, which provides a cell niche for tissue regeneration. FIG. 6 shows the percentage of cell attachment to different hyaluronic acid coatings with different molecular weights. When corneal stromal cells attach to TCPS and the interfaces of different molecular weights for one hour and eight hours, the results are not significantly different (p>0.05; n=4). Moreover, comparing TCPS with the interface of hyaluronic acid, the results show a trend that the cell attaching quantity is low between the cell and hyaluronic acid coatings because the surface of the cell is slightly negative charged and the hyaluronic acid is a polymer material with negative charges, they exclude each other to reduce the rate of cell attachment (*P<0.05; n=4). FIG. 7 (*P<0.05; n=4) illustrates corneal stromal cell growth on different hyaluronic acid coatings, and the results show that when the corneal stromal cells were cultured on HA35 coatings, the cells grow with the increase of days, and the growth rate is higher than the group cultured on TCPS. On the other hands, the corneal stromal cells cultured on HA360 and HA1500, the cell growth is stationary. The experiment proves that when the cells aggregate to form cell spheroids, the quantity of cells does not increase with time, but the cells cultured on HA35 and TCPS do.

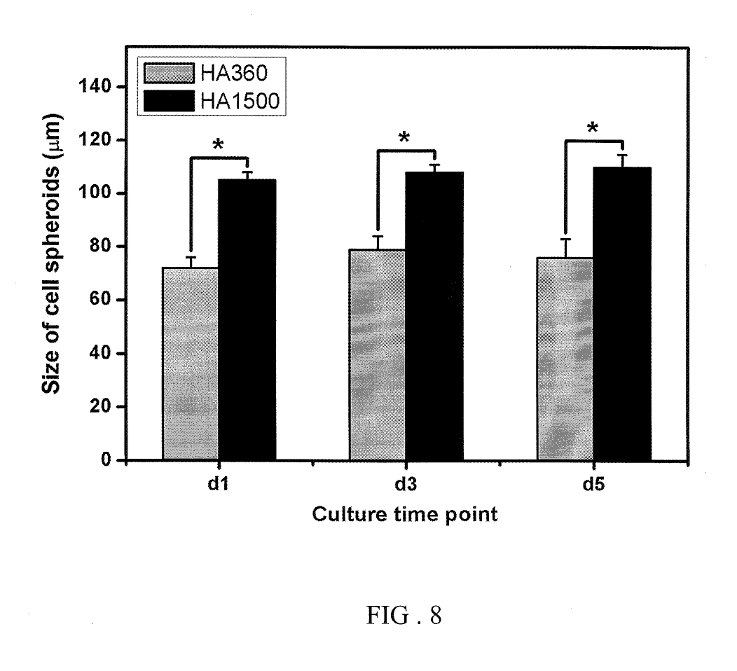

Quantitative analysis of cell spheroids: use an optical microscope to take 10 images in different areas of the corneal stromal cells at the first, third and fifth day, and analyze the number and size of the cell spheroids using Adobe image software. When the size of the cell is larger than 50 μm, it is considered a cell spheroid. FIG. 8 (*P<0.05; n=4) illustrates the size of the corneal stromal cells cultured on the hyaluronic acid coatings with high molecular weight under the optical microscope. The results show that the size of the corneal stromal cells cultured on HA1500 hyaluronic acid coatings is larger, and the sizes thereof for the first, third, and fifth day are 105±3, 108±3 and 110±5 μm. Also, the sizes on HA360 for the first, third, and fifth day are 72±4, 79±5 and 76±7

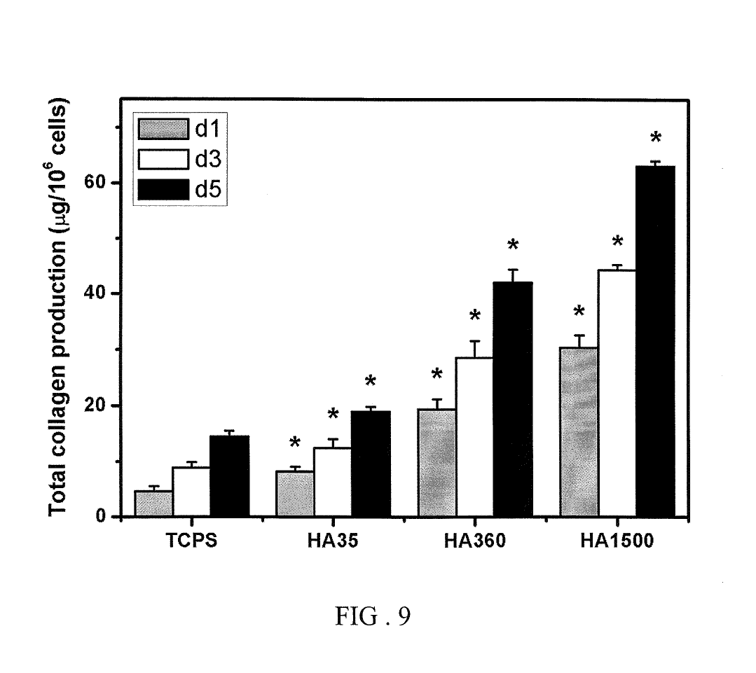

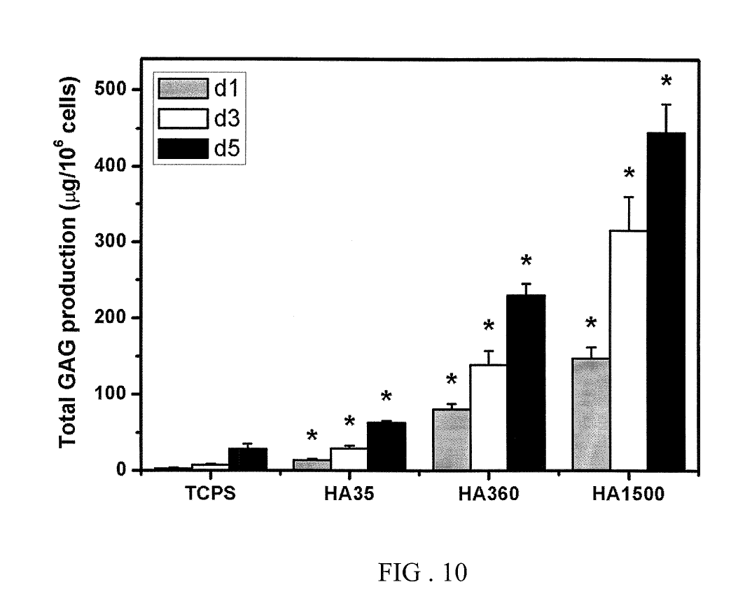

Extracellular matrix analysis: the corneal stromal cells are cultured on TCPS and hyaluronic acid coatings with different molecular weights, and the amount of collagen and GAG on the first, third and fifth day are analyzed, where the amount of hydroxyproline are used to measure the capability for the cells to secret collagen. The experimental steps are: collecting culture medium for the first, third and fifth day, boiling 6N hydrochloric acid at 110° C. for 18 hours and after it cools down, adding it to sodium chloride solution to neutralize the sample solution; adding chloramine-T buffer reagent and oxidant at room temperature for 25 minutes; and using spectrophotometer to get the adsorption value (at 550 nm wavelength) with hydroxyproline as a calibration curve for collagen analysis. The experiment will be repeated three times. Also, to analyze the capability of the corneal stromal cell to secrete GAG on different hyaluronic acid coatings with different molecular weights, cell culture medium for the first, third and fifth day are collected, mixed with DMMB reagent (40 mM NaCl; 40 mM glycine; 46 μM dimethylmethylene blue, pH 3.0), and the adsorption value is obtained from UV-Vis spectrophotometer at 525 nm wavelength using 50 μg/mL chondrotin sulfate as a calibration curve. FIGS. 9 and 10 illustrate the secretion amount of collagen and GAG on different hyaluronic acid coatings with different molecular weights. Since the corneal stromal cells cultured on TCPS and HA35 are single-layered, and on HA360 and HA1500 are aggregated to form cell spheroids, the results show that comparing to cell spheroids; single-layered cells have low secretion of collagen and GAG. As shown in FIG. 9 (*P<0.05; n=3), when the cells are cultured for five days, collagen secretion in each group is: TCPS (14.5±1.0 μg/10⁶ cells); HA35 (19.0±0.9 μg/10⁶ cells); HA360 (42.1±2.4 μg/10⁶ cells); HA1500 (63.2±0.9 μg/10⁶ cells). As can be seen in FIG. 10 (*P<0.05; n−=3), GAG secretion in each group is TCPS (28.1±7.3 μg/10⁶ cells); HA35 (63.2±2.7 μg/10⁶ cells); HA360 (230.5±15.6 μg/10⁶ cells); HA1500 (445.5±37.8 μg/10⁶ cells). In addition, the corneal stromal cells have larger cell spheroids on HA1500 hyaluronic acid coatings with larger surface roughness. The results also show that cell spheroids on HA1500 hyaluronic acid coatings have more secretion of extracellular matrix, for example, the secretion of collagen and GAG on HA1500 is 333% and 705% comparing with the secretion on HA35.

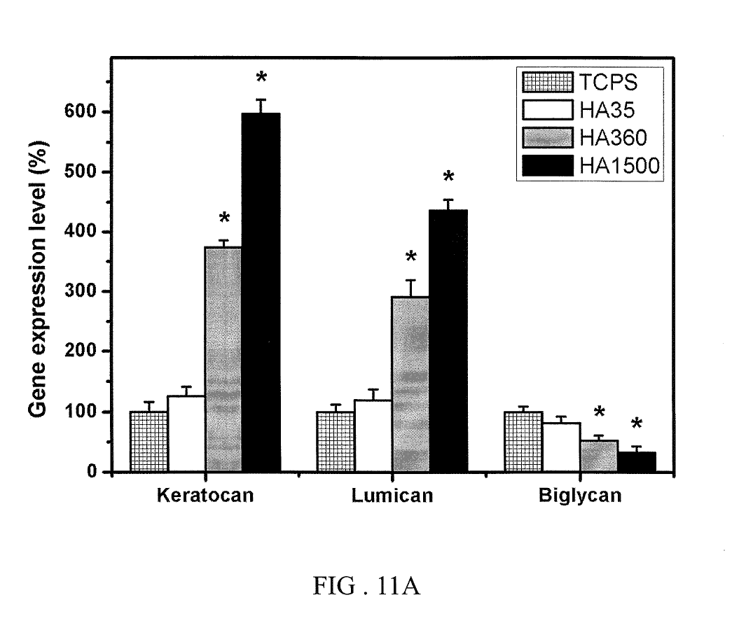

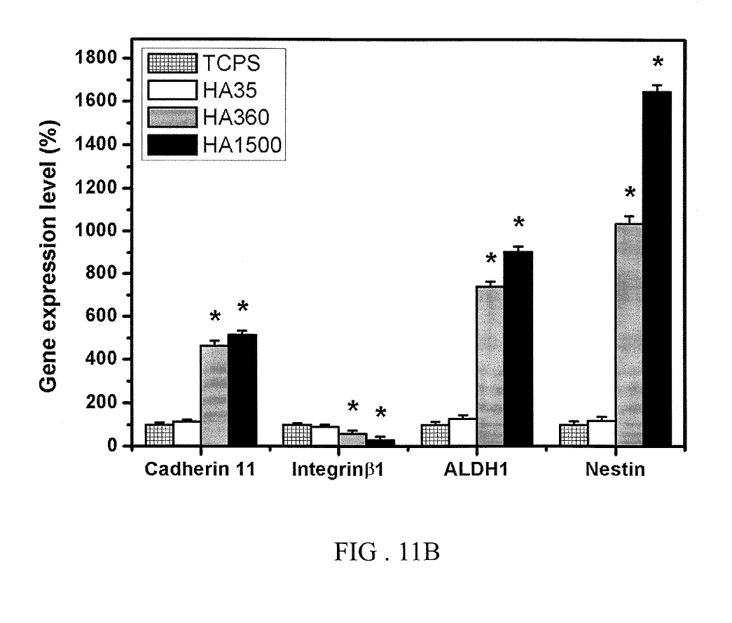

Gene expression: to assess gene expression of the corneal stromal cells, reverse transcription PCR is used for quantitative analysis. According to TRIzol standard for purifying RNA, the steps include: put one 1 mL Trizol reagent on the cells cultured on the hyaluronic acid coatings; add 200 μL Chlolofrom reagent to mix for 15 seconds at room temperature for 3 minutes; use ultra-high-speed centrifuge (12000 rpm) to centrifuge the mixture for 15 minutes; remove supernatant liquid and wash it once with 75% alcohol; dry the washed supernatant liquid for 10 minutes with air dry; use 60° C. DEPC to dissolve and determine RNA concentration with a nucleic acid analyzer. Subsequently, use SuperXcrip™ and III/RNaseOUT™ reagents to conduct reverse transcription reaction, and take 1 μg RNA, 1 μL 50 oligo-dT primer and 1 μL Annealing buffer to react for 5 minutes at 65° C. Also, add 10 μL 2× First-Strand Reaction Mix and 2 μL SuperXcrip™ and III/RNaseOUT™ Enzyme Mix to react at 50° C. for 50 minutes, and finally react at 85° C. for 5 minutes. Subsequently, take the cDNA and FastStart DNA Master SYBR Green I reagent for PCR quantitative reaction at LightCycler analyzer (95° C. for 10 seconds, 65° C. for 5 seconds, and 72° C. for 6 seconds, 45 cycles), and use GADPH as a control group. Detection genes are (keratocan, lumican, biglycan, cadherin 11, integrin beta 1, ALDH1, nestin, and GAPDH) and their gene sequences are shown as primers in table 1. FIG. 11A (*P<0.05; n=3) illustrates that the phenotypic genes (Keratocan, Lumican, Biglycan) of the corneal stromal cells are similar on TCPS and HA35. Comparing with cells cultured on HA35 coatings, cells cultured on HA360 and HA1500 have higher phenotypic gene expression levels. These results show that as compared with two-dimensional monolayered cells, the three-dimensional cell spheroids have morphological characteristics similar to those of keratocytes in vivo. Gene expression level of Keratocan on TCPS, HA35, HA360 and HA1500 are: 100±16.2%, 125±15.9%, 374.1±11.3%, 596.5±23.7%, respectively. For Lumican are: 100±12.7%, 119.2±18.0%, 291±28.4%, 436.2±18.6%, respectively. For Biglycan are: 100±9.4%, 81.5±11.6%, 52.9±8.5%, 33.1±9.9%, respectively. The results show that the corneal stromal cells can maintain cell morphology and proper phenotype when cultured on the nanoscale hyaluronic acid coatings with high roughness. To analyze cell behavior on different nanotopographies, the gene expression level of cadherin 11 (cell-cell adhesion molecule) and integrin β1 (cell-matrix adhesion molecule) is analyzed, as shown in FIG. 11B (*P<0.05, n=3). The results show that comparing with TCPS and HA35 coatings, the cells cultured on HA360 and HA1500 have higher expression amount of cadherin 11, and lower expression amount of integrin β1. Namely, when cells form spheroids, the cell-cell interaction force is higher while the cell-substrate interaction force is lower. The gene expression levels in four groups (TCPS, HA35, HA360, HA1500) regarding cadherin 11 is 100±10.9%, 113.3±9.4%, 461.8±25.1%, 513.6±20.9%, respectively. The gene expression level of integrin β1 is 100±8.1%, 90.2±11.7%, 59.5±13.3%, 28.7±16.0%. Also, the corneal stromal cells have high expression level of aldehyde dehydrogenase (ALDH) inside, and this gene expression is highly associated with the transparency of the cornea. Corneal proteins, such as ALDH, can help reduce refractive index. Experimental results show that the corneal stromal cells cultured on hyaluronic acid coatings with high molecular weights (HA360 and HA1500) have higher ALDH expression amount. Namely, when the cells are cultured on a surface with high roughness to form stationary cell spheroids, the transparency of the cell increases. On the other hands, when analyzing gene expression amount of Nestin, human corneal stromal cells have high gene expression of Nestin, which is a sign of an undifferentiated gene. Comparing with TCPS and HA35, cell cultured on HA360 and HA1500 have higher gene expression level of Nestin, meaning that corneal stromal cell spheroids have higher self-renewal capabilities. Gene expression for cells cultured on TCPS, HA35, HA360 and HA1500 are 100±15.6%, 118.8±19.1%, 1036.2±38.0%, 1647.5±32.3%.

Having described the invention by the description and illustrations above, it should be understood that these are exemplary of the invention and are not to be considered as limiting. Accordingly, the invention is not to be considered as limited by the foregoing description, but includes any equivalents.

TABLE 1

| Gene | |
|---|---|
| Accession | Forward (5′-3′) |
| Number | Reverse (5′-3′) |
| Keratocan | CTCACGTGGCTTTGATGTGT |
| XM_002711384 | GACCTTTGTGAGGCGATTGT |
| Lumican | CGAAAGCAGAGGCAAGACAGTA |
| NM_001195680 | GTATTGGCCACTGGCACCAT |
| Biglycan | CGGGAGCTTCATCTAGACAACA |
| NM_001195691 | TCGTTGACACCCACCTTGGT |
| Cadherin II | GGCTCAACATCTCTGTCTTTGCA |
| XM_002711554 | CAGCAAACTTGGGAGCATTATCA |
| Integrin β1 | AGGAGAAAATGAATGCCAAATGG |
| XM_002721189 | GGGTGCTCATTTTCCCTCATACTT |
| ALDH1 | GCAGCCATTTCTTCCCACAT |
| NM_001082013 | CCCTCTTCAGGTTGCTTTTCC |
| Nestin | TGGCACACCTCAAGATGTCTCT |
| XM_002715314 | GAAACGCAGGTCCAGCTTTG |
| GAPDH | TTGCCCTCAATGACCACTTTG |
| XM_002713034 | TTACTCCTTGGAGGCCATGTG |

### Animal Experiments

Establish animal model for impaired corneal stroma: use Pantobarbital/Rompun (2:1) to conduct general anesthesia (1.5 mL/kg of body weight) on a rabbit, and apply Alcaine on the rabbit's eye for local anesthetics. Open the rabbit's eyelid with wire lid spectrum and use 30G needle to inject 1000 CFU Staphylococcus aureus on the rabbit's right corneal stroma to induce impairment of the corneal stroma. Staphylococcus aureus does not apply to the rabbit's left eye as a control group.

Therapy model: after six hours of induced bacterial keratitis, the following four groups are used to assess the outcome of the treatment: 1. Ctrl: untreated; 2. ED: giving antibiotic therapy (eye drops administered every six hours, continue for two weeks); 3. EDCsu: administer medicine and cell suspensions (total amount of transplanted cells: 1*10⁵ cells); 4. EDCsp: administer medicine and cell spheroids (total amount of transplanted cells: 1*10⁵ cells).

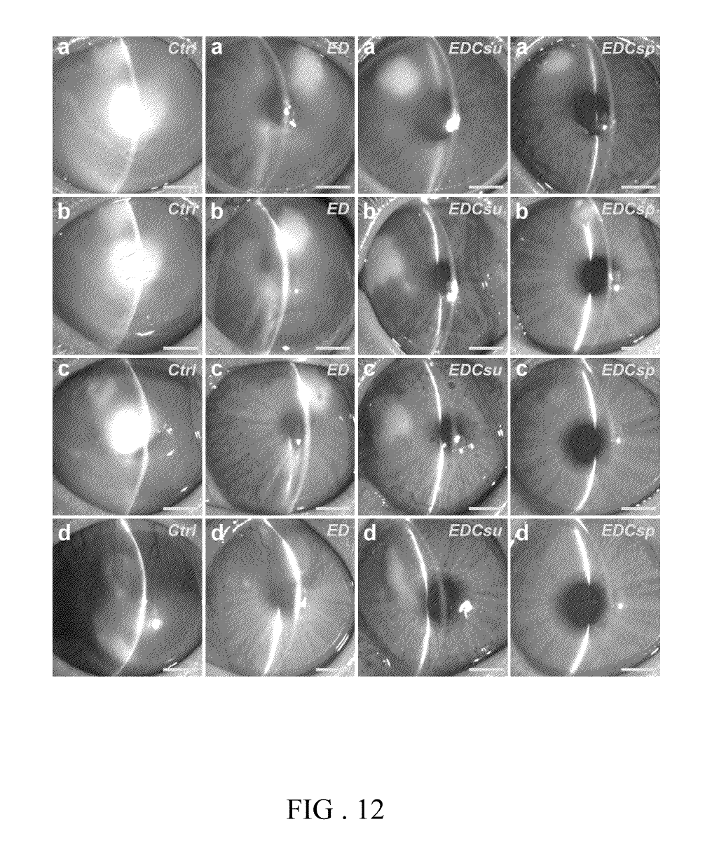

The slit lamp observation: FIG. 12 shows slit lamp observation, sub-figures from top to bottom, are 1d, 3d, 7d, 14d, observation records for four groups.

Ctrl: the control group shows the cornea without treatment after being induced. With the increase of days, severe edema, neovascularization, and scar occur.

ED: the group only subjected to antibiotic treatment. Even though the situation of fungus infection has been improved with the increase of days, corneal scar is still obvious, meaning that even though antibiotics can effectively inhibit the bacteria, but can not restore the impaired cornea to its normal situation.

EDCsu & EDCsp: comparing with the cell suspensions, cell spheroids have better scar repair results.

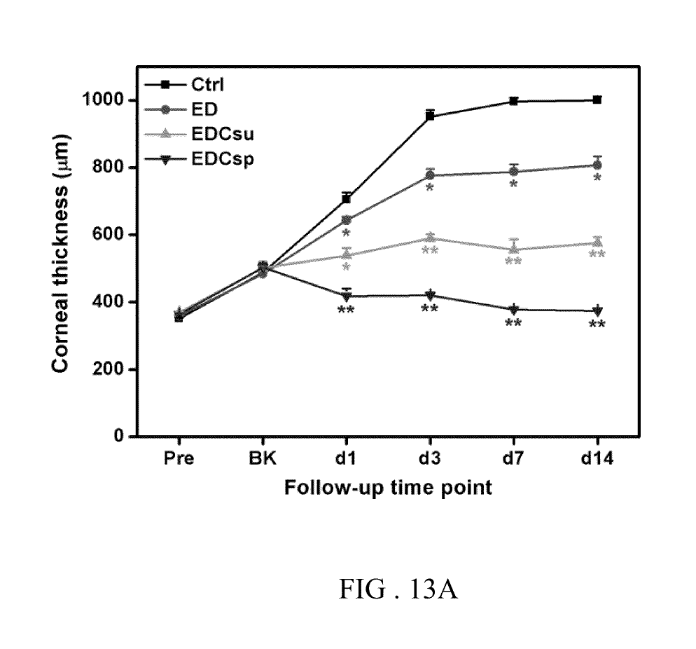

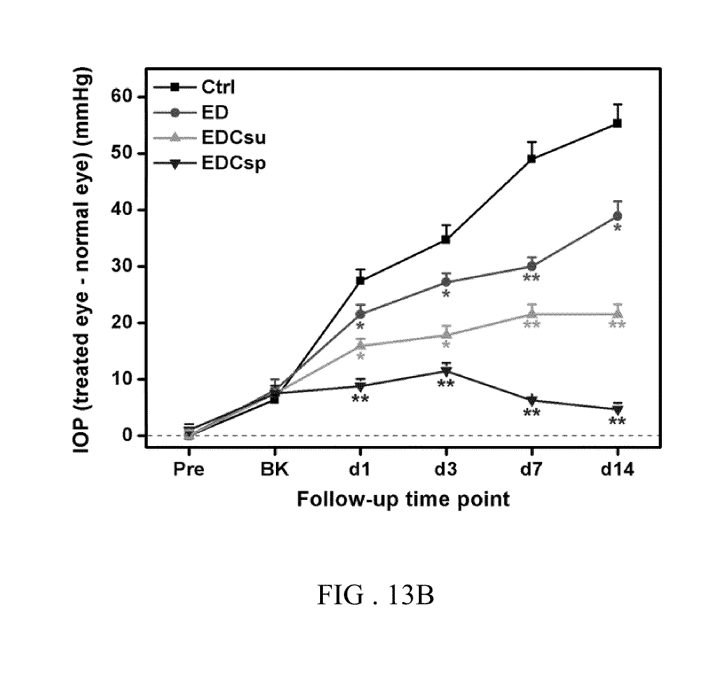

Observation of corneal thickness and intraocular pressure: FIG. 13A (P<0.05; **P<0.001; n=6) shows the observation of corneal thickness in each group at various time points. When the corneal is infected by bacteria, it would lead to iritis and corneal edema that causes the increase of corneal thickness. FIG. 13B (*P<0.05; **P<0.001; n=6) shows the intraocular pressure in each group at various time points. When the corneal stromal is damaged to cause bacterial infection, it may lead to cell inflammatory to block the trabecular meshwork resulting in edema, and the rise in intraocular pressure. The results show that the intraocular pressure in Ctrl group continued to increase with the increase of days. The use of antibiotics does not provide good treatment to lower intraocular pressure. Comparing with the group of cell suspensions, the intraocular pressure in the spheroids treatment group does not increase continuously, and remains stable.

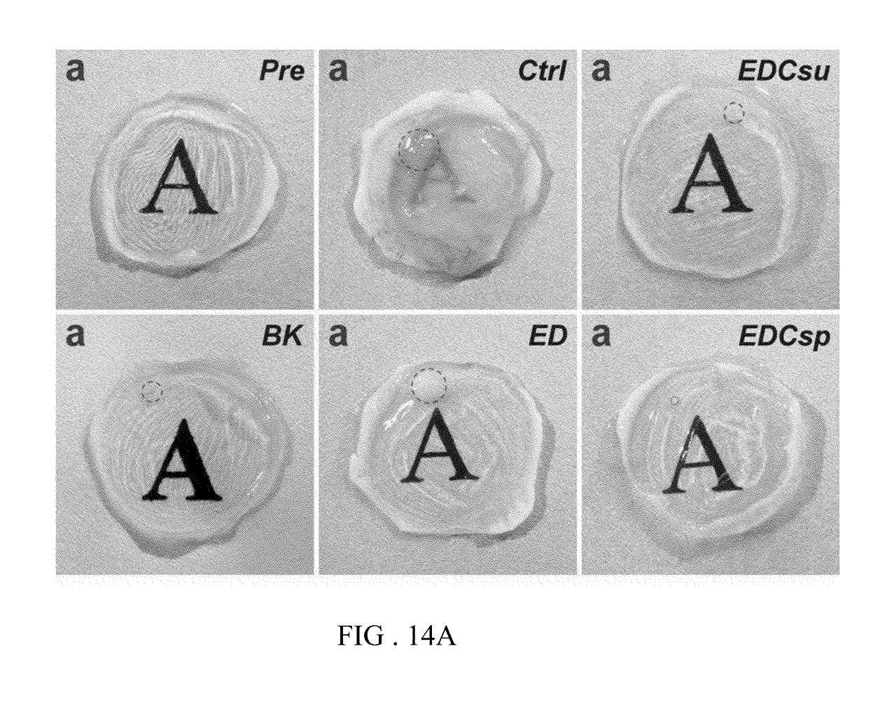

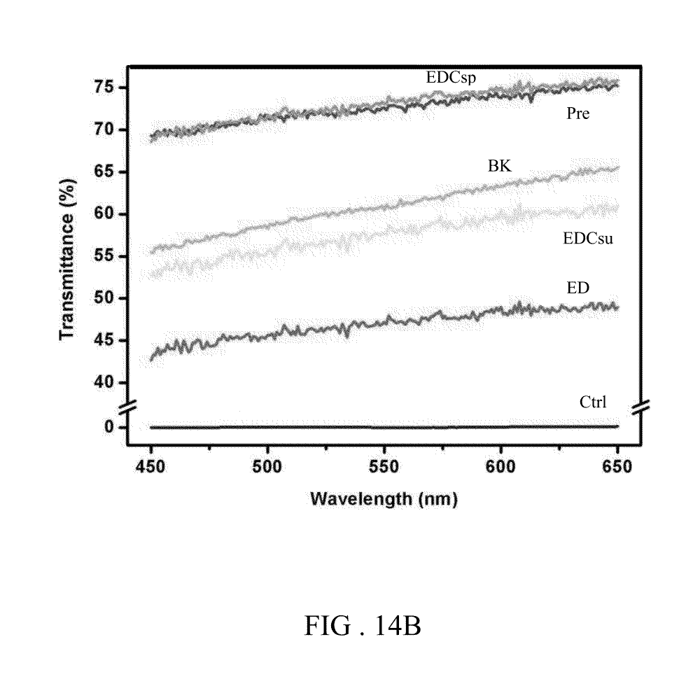

When the corneal is infected by bacteria, the corneal transparency will be reduced due to inflammation, edema and other symptoms. FIG. 14A shows corneal analysis diagrams of the six groups. The experimental animals are sacrificed to observe the corneal transparency (appearance and visible light transmittance) in the following six categories: 1. Pre: before induced; 2. Bk: after induced; 3. Ctrl: no treatment after 14 days; 4. ED: administer antibiotic treatment for 14 days; 5. EDCsu: administer medicine and the cell suspensions for 14 days; 6. EDCsp: administer medicine and spheroids for 14 days. When observing the corneal appearance of transparency, the corneal tissue scar is more obvious in the untreated group than in the group treated with antibiotics. In addition, comparing with cell suspensions, cell spheroid group has better repairing results. FIG. 14B (n=6) shows corneal optical transmittance in six groups, using UV/visible spectrometer in the visible range to observe corneal penetration. The repair is much better in the spheroid group; the result shows that the degree of light penetration can almost recover to the degree before being induced.

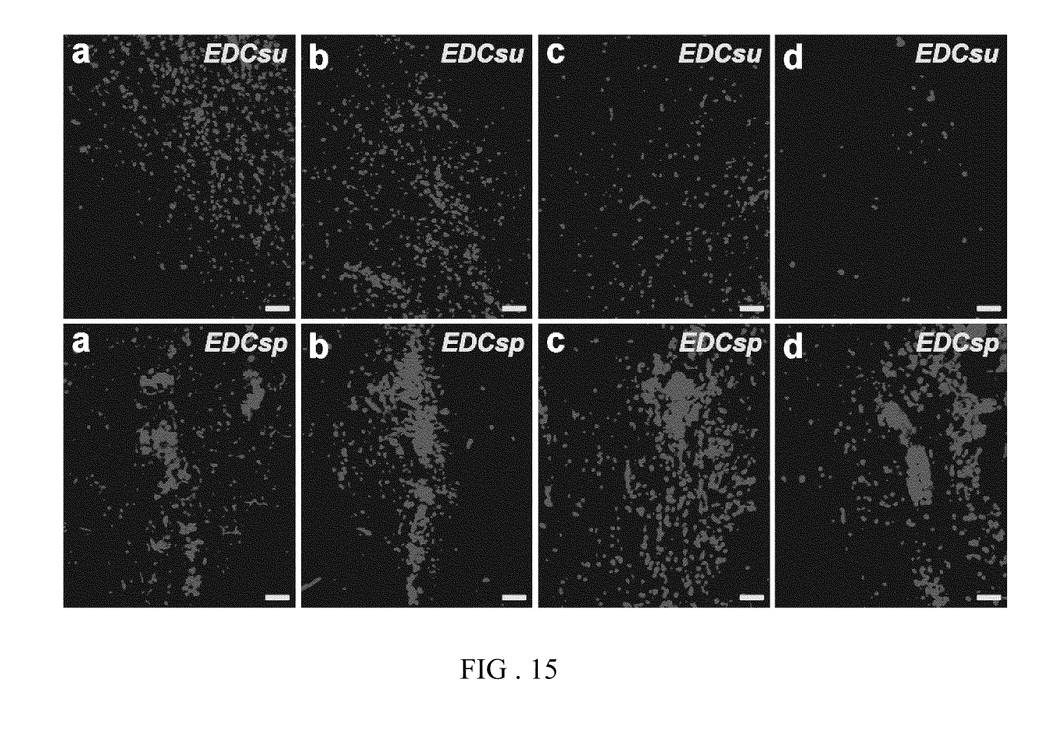

Assessment of spheroids and cell suspensions in the body: the red fluorescent dye PKH26 labels the membrane surface. First, the spheroids and cell suspensions are each placed in an eppendorf, and add 0.6 mL of Diluent C and 1 μL PKH 26 dye stock. Also, 200 μL of dye is added to each eppendorf, which is incubated at 37° C. for 5 minutes, and then centrifuged at 200 rpm for 1 minute. The supernatant of each eppendorf is removed and dissolved in 10 μL medium. FIG. 15 shows distribution of the corneal stromal cells observed by fluorescence microscope. From left to right are the cases of PKH26 staining for 0, 1, 7, 14 days, and the gray dots are cells, which shows that the retention ability of the spheroids in the eye is higher.

According to the results of aforementioned in vivo animal experiments, when the corneal stromal cell spheroids are injected via a small needle to the damaged corneal stroma, the results show that the corneal thickness, intraocular pressure, light penetration and corneal scars are well recovered to the normal cornea after receiving treatment for 14 days. In the present invention, the corneal stromal cell spheroids can stay longer in the damaged area, and can effectively repair the corneal stroma. It can be applied to the treatment of eye diseases include at least chemical/thermal burns, bacterial keratitis, herpetic keratitis, severe dry eye, ocular rosacea, Stevens-Johnson syndrome, and neuropathic ulcers.

1. (canceled)

2. A method of treating eye diseases comprising:
preparing nanoscale hyaluronic acid coatings of different high molecular weights, wherein the range of molecular weights of the hyaluronic acid coatings is between 360,000 and 1,500,000 Daltons;
analyzing surface roughness, surface potential and surface contact angle of each hyaluronic acid coating;
culturing corneal stromal cells on said nanoscale hyaluronic acid coatings and conducting cell adhesion analysis, proliferation analysis, quantitative spheroid size and number analysis, extracellular matrix analysis and gene expression analysis;
maintaining cell aggregation;
forming cell spheroids cultured in a serum-free manner on the nanoscale hyaluronic acid coatings with the different high molecular weights; and
injecting the cell spheroids to a corneal stroma.

3. The method of claim 2, wherein the range of the surface charge of the hyaluronic acid coatings is between −14.8 and −48.2 mV.

4. The method of claim 2, wherein the surface roughness of the hyaluronic acid coatings is higher than 0.9 nanometer.

5. The method of claim 2, wherein the contact angle between the hyaluronic acid coatings and deionized water is smaller than 49.6 degrees.

6. The method of claim 2, wherein the size of the spheroid is between 68 and 115 micrometers in diameter.

7. The method of claim 2, wherein the cell spheroids secrete an extracellular matrix after formation, and wherein the cell spheroids cultured on the nanoscale hyaluronic acid coatings with the different higher molecular weights have higher secretion capability.

8. The method of claim 7, wherein the extracellular matrix at least includes collagen and glycosaminoglycans (GAG).

9. The method of claim 8, wherein the range of collagen secretion is between 39.7 and 64.1 (μg/10⁶ cells).

10. The method of claim 8, wherein the range of GAG secretion is between 214.9 and 483.3 (μg/10⁶ cells).

11. The method of claim 2, wherein the cell spheroids maintain a mitotically quiescent state.

12. The method of claim 2, wherein the gene expression level of the corneal stromal cells on the nanoscale hyaluronic acid coatings with the different molecular weights is quantitatively analyzed with a reverse transcription polymerase chain reaction.

13. The method of claim 2, wherein the wherein the proper phenotype of cell spheroids is verified by gene expression levels of keratocan, lumincan, and biglycan.

14. The method of claim 2, wherein the transparency of the cell spheroids is accessed by gene expression level of aldehyde dehydrogenase (ALDH).

15. The method of claim 2, wherein the corneal stroma include eye diseases including chemical/thermal burns, bacterial keratitis, herpetic keratitis, severe dry eye, ocular rosacea, Stevens-Johnson syndrome, and neuropathic ulcers.
